(19) United States
(12) Patent Application Publication
(10) Pub. No.: US 2010/0273162 A1
(43) Pub. Date: Oct. 28, 2010

(54) RAPID DETECTION OF MICROORGANISMS

(76) Inventor: Sailaja Chandrapati, Woodbury, MN (US)

Correspondence Address: 3M INNOVATIVE PROPERTIES COMPANY, PO BOX 33427, ST. PAUL, MN, 55133-3427, US

(21) Appl. No.: 12/676,613

(22) PCT Filed: Oct. 15, 2008
(86) PCT No.: PCT/US08/79924

§ 371 Date: Mar. 5, 2010

Related U.S. Application Data
(60) Provisional application No. 60/980,722, filed on Oct. 17, 2007.

(51) Int. Cl. C12Q 1/68; C12M 1/34

(52) U.S. Cl. 435/6; 435/287.2

(57) ABSTRACT

Methods for rapidly detecting Enterobacteriaceae and Micrococcaceae microorganisms utilizing non-amplified nucleic acids, acridiniu labeled ONA probes, and selective growth media are described, particularly for specific microbial species related to the food science industry and public health. Articles of manufacture that include reagents for detecting multiple microorganisms simultaneously are also described.

### TECHNICAL FIELD

This document relates to methods and materials for detecting microorganisms. More specifically, this document relates to methods for detecting microorganisms by rapidly enriching for the microorganisms and using a non-amplified nucleic acid based test to detect the microorganisms.

### BACKGROUND

To prevent the transmission of food-borne pathogens, manufacturers and/or processors of food products routinely test samples to identify contaminated products before product is released to the consumer. The presence of a sufficient number of pathogens can result in the contamination of a food product and, additionally, if consumed by a human or animal, result in food-borne illness. In the United States, the number of cases of food poisoning associated with the consumption of contaminated food products is conservatively estimated to be in the multi-millions per year. While most human cases of bacterial food poisoning only result in acute symptomatic disease (e.g., nausea, vomiting, diarrhea, chills, fever, and exhaustion), death can occur in infants, the elderly, pregnant women, and those with immunocompromised systems.

Typical methods of detecting pathogens include pre-enrichment where the food sample is enriched in a non-selective medium to restore injured bacterial cells to a stable physiological condition, selective enrichment where growth-promoting substances and selective inhibitory reagents are added to the medium to promote the growth of selected pathogenic microorganisms while restricting the proliferation of most other bacteria, and detecting any pathogenic microorganisms by biochemical assays, immunoassays, polymerase chain reaction (PCR), or serological techniques. These methods can take 24-72 hours to complete.

### SUMMARY

Disclosed is a rapid method for detecting microorganisms in a sample. The method includes an enrichment step that can be performed in the same vessel used to homogenize the sample. Microorganisms can be detected in the enriched sample by a variety of methods, including non-amplified nucleic acid-based tests such as the hybridization protection assay. The methods described herein can be used to detect low levels of pathogens within food matrices in less than 18 hours.

In one aspect, a method is disclosed for detecting a target microorganism in a sample (e.g., a food sample such as a dairy product, a meat, a vegetable, or a seafood). The method includes homogenizing a sample in a vessel (e.g., a bag), wherein the vessel includes a growth medium; incubating the homogenized sample in the vessel to enrich for target microorganisms if present in the sample; and detecting a non-amplified nucleic acid of the target microorganism. The target microorganism can be detected in a mixture that includes nucleic acid from a non-target microorganism. The target microorganism can be selected from the group consisting of Enterobacteriaceae and Micrococcaceae. For example, the target microorganism can be selected from the group consisting of Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Clostridium spp., Campylobacter spp., Vibrio spp., and Corynebacteria spp.

The detecting step can include lysing microorganisms in the sample; hybridizing a nucleic acid probe to a target nucleic acid sequence of the target microorganism to form a probe:target complex, wherein the probe includes a label that is stabilized by the complex; selectively degrading the label present in unhybridized probe, and detecting the presence or amount of stabilized label as a measure of the presence or amount of the target nucleic acid sequence in the sample. The probe can be labeled with an acridinium ester. The probe can hybridize to ribosomal RNA of the target microorganism.

The growth medium can include a growth inhibitor for non-target microorganisms. The growth inhibitor can be selected from the group consisting of bile salts, sodium deoxycholate, sodium selenite, sodium thiosulfate, lithium chloride, potassium tellurite, sodium tetrathionate, sodium sulphacetamide, mandelic acid, selenitecysteine tetrathionate, sulphamethazine, brilliant green, malachite green, crystal violet, Tergitol 4, sulphadiazine, amikacin, aztreonam, naladixic acid, acriflavine, polymyxin B, novobiocin, and alafosfalin.

The incubating step can be performed at 30° C. to 45° C. for 10 to 18 hours. For example, the incubating step can be performed at 35° C. to 42° C. for 10 to 18 hours. The growth medium can include nutrients that allow the growth of the target microorganism to a minimum level 10⁴ cfu/mL. The growth medium can include nutrients that support the growth of more than one target microorganism.

Also featured is an article of manufacture for detecting a microorganism. The article of manufacture includes a multi-well solid substrate, wherein each well of the solid substrate is coated with a lysing reagent and a nucleic acid probe. In some embodiments, the substrate further is coated with a selection agent. The article of manufacture further can include a homogenization vessel, where the homogenization vessel includes a growth medium coated on the inner surface of the vessel. The coating can be a dried coating. The homogenization vessel further can include a growth inhibitor for a non-target microorganism coated on the inner surface of the vessel. The multi-well solid substrate can be a 96-well plate, a 384-well plate, or a microfluidic sample processing device. The article of manufacture can include a plurality of multi-well substrates, wherein each multi-well substrate is targeted to a different microorganism.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

In general, materials and methods are disclosed for detecting microorganisms from a sample. The methods disclosed herein include homogenizing the sample in a vessel that includes a growth medium, and, after incubating the homogenized sample to enrich for the microorganisms, detecting the microorganism using a nucleic acid based test such as the hybridization protection assay. Such methods allow the user to detect the microorganisms with minimal handling.

The methods disclosed herein can be used for detecting microorganisms from a variety of food and non-food samples that contain a mixed population of microorganisms. “Food” refers to a solid, liquid or semi-solid comestible composition. Examples of foods include, but are not limited to, meats, poultry, eggs, fish, seafood, vegetables, fruits, prepared foods (e.g., soups, sauces, pastes), grain products (e.g., flour, cereals, breads), canned foods, cheese, milk, infant formula (e.g., powdered or liquid infant formula), other dairy products (e.g., cheese, yogurt, sour cream), fats, oils, desserts, condiments, spices, pastas, beverages, water, other suitable comestible materials, and combinations thereof.

“Nonfood” refers to sources of interest that do not fall within the definition of “food.” Particularly, nonfood sources can include, but are not limited to, substances that are generally not comestible and that may be categorized as one or more of a cell lysate, whole blood or a portion thereof (e.g., serum), other bodily fluids (e.g., saliva, sweat, sebum, urine), feces, cells, tissues, organs, plant materials, wood, soil, sediment, animal feed, animal carcasses, vegetable rinses, process water, medicines, cosmetics, environmental sampling devices (e.g., sponges or swabs), and other suitable non-comestible materials, and combinations thereof.

Microorganisms of particular interest include prokaryotic and eukaryotic organisms, particularly Gram positive bacteria, Gram negative bacteria, fungi, protozoa, mycoplasma, yeast, viruses (e.g., HIV and HPV), and lipid-enveloped viruses. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Campylobacter, Vibrio spp., Clostridium spp., Corynebacteria spp. Particularly virulent organisms include Escherichia coli (e.g., E. coli O157:H7), Salmonella enteritidis, and Salmonella typhi.

Typically, a sample (e.g. a food sample) is placed in a vessel (e.g., a bag, tube, flask, or bottle) that contains a growth medium. The sample can be homogenized to mix the sample and growth medium, and to release any microorganisms that may be contained within a solid or semi-solid sample. Techniques for homogenization can include stirring, mixing, agitating, blending, or vortexing. For example, a blender can be used to homogenize samples at 10,000 to 12,000 rpm as recommended by the Food and Drug Administration, “Food Sampling and Preparation of Sample Homogenate”, Chapter 1; FDA Bacteriological Manual, 8th Ed.; 1998, section 1.06. A “stomaching” device can be used that mixes a source and diluents in a bag through the use of two paddles in a kneading-type action. See, for example, U.S. Pat. No. 3,819,158. An oscillating device known as the PULSIFIER® is described in U.S. Pat. No. 6,273,600, which employs a bag placed inside an agitating metal ring. Another technique, vortexing for analyte suspension, has been described in U.S. Pat. No. 6,273,600. See also U.S. Patent Application Publication No. 2007/026931 A-1, for a device that can mix a sample and growth medium.

A suitable growth medium contains nutrients that allows rapid recovery of potentially injured target microorganisms and growth of a target microorganism to a minimum of 10⁴ colony forming units per milliliter (cfus/mL). Non-limiting examples of a growth medium include Tryptic Soy Broth (TSB), Buffered Peptone Water (BPW), Universal Pre-enrichment Broth (UPB), Listeria Enrichment Broth (LEB), or other general, non-selective, or mildly selective media known to those skilled in the art. The medium can include nutrients that that support the growth of more than one target microorganism.

Typically, the growth medium includes a growth inhibitor of non-target microorganisms. For example, one or more of bile salts, sodium deoxycholate, sodium selenite, sodium thiosulfate, lithium chloride, potassium tellurite, sodium tetrathionate, sodium sulphacetamide, mandelic acid, selenitecysteine tetrathionate, sulphamethazine, brilliant green, malachite green, crystal violet, Tergitol 4, sulphadiazine, amikacin, aztreonam, naladixic acid, acriflavine, polymyxin B, novobiocin, and alafosfalin can be used to inhibit the growth of non-target microorganisms.

In some embodiments, the vessel contains liquid growth medium. In other embodiments, the inner surface of the vessel is coated with the growth medium and/or growth inhibitor. The coating can be dried to provide a dry medium on the inner surface of the vessel. The dry medium can be rehydrated upon adding the sample and an appropriate buffer.

After homogenization, the vessel is incubated for a time and temperature sufficient for the growth of at least 10⁴ cfus/mL of the target microorganism. For example, the vessel can be incubated at 30° C. to 45° C. for 10 to 18 hours. Incubation temperatures of 35° C. to 42° C. are particularly useful.

### Detecting Non-Amplified Nucleic Acid

Microorganisms can be detected using, for example, a hybridization protection assay (HPA). In this method, microorganisms can be lysed to release nucleic acid. For example, a detergent such as sodium dodecyl sulfate (SDS) or sodium N-lauroyl sarcosine, or an enzyme such as lysozyme or lysostaphin can be used to lyse the cells. Alternatively, a change in temperature ,pH, or osmotic pressure can be used to lyse the cells.

An oligonucleotide probe can be hybridized to a target nucleic acid sequence of the target microorganism to form a probe:target complex. As used herein, the term “oligonucleotide” refers to an oligomer or polymer of ribonucleic acid (RNA) or deoxyribonucleic acid (DNA), or analogs thereof. Nucleic acid analogs can be modified at the base moiety, sugar moiety, or phosphate backbone to improve, for example, stability, hybridization, or solubility of a nucleic acid. Modifications at the base moiety include substitution of deoxyuridine for deoxythymidine, and 5-methyl-2′-deoxycytidine and 5-bromo-2′-deoxycytidine for deoxycytidine. Other examples of nucleobases that can be substituted for a natural base include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone (e.g., an aminoethylglycine backbone) and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone. See, for example, U.S. Pat. Nos. 4,469,863, 5,235,033, 5,750,666, and 5,596,086 for methods of preparing oligonucleotides with modified backbones.

The oligonucleotide probe can hybridize with any portion of a nucleic acid from the target microorganism. For example, an oligonucleotide can hybridize with a nucleic acid encoding a cell-wall protein or an internal cell component, such as a membrane protein, transport protein, or enzyme. In some embodiments, the oligonucleotide hybridizes with ribosomal RNA (rRNA) or a mRNA of a target microorganism. See, for example, U.S. Pat. No. 4,851,330. For example, the oligonucleotide can hybridize with a 16S, 23S, or 5S rRNA. Hybridization to rRNA can increase the sensitivity of the assay as most microorganisms contain thousands of copies of each rRNA. For example, E. coli contains about 10⁴ copies of each rRNA subunit.

The oligonucleotide probe typically is labeled with a molecule that is stabilized by the probe:target hybrid complex. For example, the oligonucleotide probe can be labeled with the highly chemiluminescent acridinium ester (AE) molecule. Alkaline hydrolysis of the ester bond of AE renders it permanently non-chemiluminescent. Hydrolysis of the ester bond of AE is rapid when the probe is single-stranded, i.e., not hybridized with its target. In contrast, hydrolysis of the AE bond is greatly reduced when the probe is hybridized with its target. As such, the oligonucleotide probe can be hybridized with its target nucleic acid under non-hydrolyzing conditions. After hybridization, the label present in unhybridized probe can be selectively degraded by adjusting the pH of the solution such that it is mildly alkaline, e.g., pH 7 to 11. See, for example, Nelson et al. (1996), Nucleic Acids Res. 24(24):4998-5003.

Oligonucleotide probes can be between 10 and 75 (e.g., 10-14, 15-30, 25-50, 30-45, 33-40, 20-30, 31-40, 41-50,or 51-75) nucleotides in length. It is understood in the art that the sequence of an oligonucleotide need not be 100% complementary to that of its target nucleic acid in order for hybridization to occur. Rather, hybridization can occur when the oligonucleotide has at least 80% (e.g., at least 85%, 90%, 95%, 99%, or 100%) sequence identity to the complement of its target sequence. Hybridization of the oligonucleotide to its target can be detected based on the chemiluminescence observed after adjusting the pH to mildly alkaline conditions. If hybridization occurs, chemiluminescence will be observed. If hybridization does not occur, the ester bond of the AE molecule will be hydrolyzed and chemiluminescence will not be observed or will be measurably reduced.

The percent identity of a nucleic acid sequence can be determined as follows. First, a nucleic acid sequence is compared to a target nucleic acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from Fish & Richardson's web site (World Wide Web at “fr” dot “com” slash “blast”), the U.S. government's National Center for Biotechnology Information web site (World Wide Web at “ncbi” dot “nlm” dot “nih” dot “gov”), or the State University of New York at Old Westbury Library (QH 497.m6714). Instructions explaining how to use the Bl2seq program can be found in the readme file accompanying BLASTZ.

Bl2seq performs a comparison between two sequences using the BLASTN algorithm. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default settings. For example, the following command can be used to generate an output file containing a comparison between two sequences: C:\Bl12seq -i c:\seq1.txt -j c:\seq2.txt -p blastn -o c:\output.txt -q −1-r 2. If the first nucleic acid sequence shares homology with any portion of the second nucleic acid sequence, then the designated output file will present those regions of homology as aligned sequences. If the first nucleic acid sequence does not share homology with any portion of the second nucleic acid sequence, then the designated output file will not present aligned sequences.

Once aligned, a length is determined by counting the number of consecutive nucleotides from the first nucleic acid sequence presented in alignment with sequence from the second nucleic acid sequence. A matched position is any position where an identical nucleotide is presented in both the target and mammalian sequence. Gaps presented in the first sequence are not counted since gaps are not nucleotides or amino acid residues. Likewise, gaps presented in the second sequence are not counted.

The percent identity over a determined length is determined by counting the number of matched positions over that length and dividing that number by the length followed by multiplying the resulting value by 100. For example, if (1) a 300 amino acid target sequence is compared to a reference sequence, (2) the Bl2seq program presents 200 consecutive amino acids from the target sequence aligned with a region of the reference sequence, and (3) the number of matches over those 200 aligned amino acids is 180, then that 300 amino acid target sequence contains an amino acid segment that has a length of 200 and a percent identity over that length of 90 (i.e., (180÷200)×100=90).

It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It is also noted that the length value will always be an integer.

Methods for synthesizing oligonucleotides are known. Typically, an automated DNA synthesizer, such as available from Applied Biosystems (Foster City, Calif.), is used. Once an oligonucleotide is synthesized and any protecting groups are removed, the oligonucleotide can be purified (e.g., by extraction and gel purification or ion-exchange high performance liquid chromatography (HPLC)) and the concentration of the oligonucleotide can be determined (e.g., by measuring optical density at 260 nm in a spectrophotomer).

An oligonucleotide can be labeled with an AE molecule during synthesis of the oligonucleotide or can be attached after synthesis. A linker molecule can be used to attach an AE molecule to an oligonucleotide using techniques known in the art. Typically, abasic linker-arm chemistry is used as set forth, for example, in U.S. Pat. No. 6,004,745 and WO 89/02933. For example, an amine-terminated linker can be incorporated at a predetermined position in an oligonucleotide during synthesis of the oligonucleotide using abasic linker arm chemistry. After purification of the oligonucleotide, the AE molecule can be attached via the amine-terminated linker. See, for example, Nelson et al. (1996), Nucleic Acids Res. 24(24):4998-5003.

The presence, absence, or amount of unmodified label can be assessed using a luminometer (e.g., LEADER® luminometer from Gen-Probe Incorporated, San Diego, Calif. or the BacLite3 luminometer from 3M, St. Paul, Minn., or the LUMIstar Galaxy luminometer from BMG, Durham, N.C.). Luminometers such as the BacLite3 luminometer and LUMIstar Galaxy luminometer have reagent dispensing capability and temperature control are particularly useful for automating the methods disclosed herein. Such luminometers can be programmed to dispense, in a predetermined order, reagents for lysing, hybridization, and detection, and allow for incubation. Automated reagent dispensing minimizes contamination issues encountered within a moist environment such as a water bath in addition to enhancing the user friendliness of the test system. It is understood that the present method is not limited by the device used to detect the label on the oligonucleotide probe.

### Articles of Manufacture

Reagents for performing the methods described herein can be combined with packaging material and sold as a kit for detecting microorganisms from samples. For example, a kit can include a multi-well substrate such as a 96-well or 384-well plate and lysing reagent, oligonucleotide probe, and a selection agent. In other embodiments each well of the substrate is coated with a lysing reagent and the desired oligonucleotide probe. In other embodiments, each well can be coated with a lysing reagent, the desired oligonucleotide probe, and a selection agent. A multi-well substrate also can be a micro reaction vessel system (e.g., microfluidic reagent card). See, for example, a sample processing device of U.S. Pat. No. 6,627,159.

In other embodiments, a kit includes one or more additional multi-well solid substrates, wherein each substrate, well, or group of wells, is targeted to a different microorganism. For example, an article of manufacture can include 2, 3, 4, 5, 6, 7, 8, 9 or 10 multi-well substrates such that multiple microorganisms can be detected simultaneously. Thus, one multi-well substrate can be coated with a lysing reagent and oligonucleotide probe for one microorganism (e.g., E. coli) and another multi-well substrate can be coated with a lysing reagent and oligonucleotide probe for a different microorganism (e.g., Salmonella). Such substrates can be in a strip format, wherein each strip contains reagents for detecting a particular microorganism.

An article of manufacture or kit further can include a homogenization vessel that includes a growth medium and/or growth inhibitor coated on its inner surface. Articles of manufacture also may include reagents for carrying out the methods disclosed herein (e.g., buffers, control nucleic acids, sterile water, or other useful reagents for performing hybridization protection assays). Articles of manufacture further can include a package label or insert with instructions for detecting a particular microorganism or combination of microorganisms. Components and methods for producing articles of manufactures are well known.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a target microorganism in a sample, said method comprising:
a) homogenizing a sample in a vessel, wherein said vessel comprises a growth medium;
b) incubating said homogenized sample in said vessel to enrich for target microorganisms if present in said sample; and
c) detecting a non-amplified nucleic acid of said target microorganism.

2. The method of claim 1, wherein said target microorganism is selected from the group consisting of Enterobacteriaceae and Micrococcaceae.

3. The method of claim 1, wherein said target microorganism is selected from the group consisting of Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Campylobacter spp., Vibrio spp., Clostridium spp., and Corynebacteria spp.

4. The method of claim 1, wherein said detecting step comprises i) lysing microorganisms in said sample; ii) hybridizing a nucleic acid probe to a target nucleic acid sequence of said target microorganism to form a probe:target complex, wherein said probe comprises a label that is stabilized by said complex; iii) selectively degrading said label present in unhybridized probe; and iv) detecting the presence or amount of stabilized label as a measure of the presence or amount of said target nucleic acid sequence in said sample.

5. The method of claim 4, wherein said probe is labeled with an acridinium ester.

6. The method of claim 4, wherein said probe hybridizes to ribosomal RNA of said target microorganism.

7. The method of claim 1, wherein said growth medium comprises a growth inhibitor for non-target microorganisms.

8. The method of claim 7, wherein said growth inhibitor is selected from the group consisting of bile salts, sodium deoxycholate, sodium selenite, sodium thiosulfate, lithium chloride, potassium tellurite, sodium tetrathionate, sodium sulphacetamide, mandelic acid, selenitecysteine tetrathionate, sulphamethazine, brilliant green, malachite green, crystal violet, Tergitol 4, sulphadiazine, amikacin, aztreonam, naladixic acid, acriflavine, polymyxin B, novobiocin, and alafosfalin.

9. The method of claim 1, wherein said incubating step is performed at 30° C. to 45° C. for 10 to 18 hours.

10. The method of claim 1, wherein said incubating step is performed at 35° C. to 42° C. for 10 to 18 hours.

11. The method of claim 1, wherein said growth medium comprises nutrients that allow the growth of the target microorganism to a minimum level 10⁴ cfu/mL.

12. The method of claim 1, wherein said growth medium comprises nutrients that support the growth of more than one target microorganism.

13. The method of claim 1, wherein said sample is a food sample.

14. The method of claim 13, wherein said food sample is a dairy product, a meat, a vegetable, or a seafood.

15. The method of claim 1, wherein said vessel is a bag.

16. An article of manufacture for detecting a microorganism, said article of manufacture comprising a multi-well solid substrate, wherein each well of said solid substrate is coated with a lysing reagent and a nucleic acid probe.

17. The article of manufacture of claim 16, further comprising a homogenization vessel, said homogenization vessel comprising a growth medium coated on the inner surface of said vessel.

18. The article of manufacture of claim 17, wherein said coating is a dried coating.

19. The article of manufacture of claim 17, wherein said homogenization vessel further comprises a growth inhibitor for a non-target microorganism coated on the inner surface of said vessel.

20. The article of manufacture of claim 16, said article comprising a plurality of multi-well substrates, wherein each multi-well substrate is targeted to a different microorganism.
